(19) United States
(12) Patent Application Publication
(10) Pub. No.: US 2014/0234288 A1
(43) Pub. Date: Aug. 21, 2014

(54) Lysosomal Acid Lipase Therapy for NAFLD and Related Diseases

(71) Applicant: Children's Hospital Medical Center, Cincinnati, OH (US)

(72) Inventor: Gregory A. Grabowski, Lexington, MA (US)

(73) Assignee: CHILDREN'S HOSPITAL MEDICAL CENTER, Cincinnati, OH (US)

(21) Appl. No.: 14/245,042

(22) Filed: Apr. 4, 2014

Related U.S. Application Data
(63) Continuation of application No. 12/065,975, filed on Aug. 4, 2008.
(60) Provisional application No. 60/715,036, filed on Sep. 8, 2005.

(51) Int. Cl. A61K 38/46

(52) U.S. Cl. CPC A61K 38/465
USPC 424/94.6

(57) ABSTRACT

The present invention comprises methods and compositions for the treatment or alleviation of NAFLD (non-alcoholic fatty liver disease) and those conditions associated with NAFLD, including fatty liver disease, nonalcoholic steatohepatitis (NASH) and cirrhosis through the use of pharmaceutical formulations of lysosomal acid lipase or related proteins and/or polypeptides. This invention is also directed to a combination therapy treatment for treating The Metabolic Syndrome. As part of a combination therapy regime for the treatment of The Metabolic Syndrome, pharmaceutical formulations of lysosomal acid lipase or related proteins and/or polypeptides are used as part of the combination therapy regime for treating NAFLD (and NASH), which comprises one of the conditions constituting The Metabolic Syndrome.

### FIELD OF INVENTION

The present invention relates to the use of lipid hydrolyzing proteins and/or polypeptides, such as lysosomal acid lipase (LAL), for the treatment and/or prevention of non-alcoholic fatty liver disease (NAFLD), which includes NASH (nonalcoholic steatohepatitis).

### BACKGROUND

NASH is a disease of the liver characterized by inflammation and damage to the liver cells. Typically, NASH and related diseases, such as NAFLD (Nonalcoholic Fatty Liver Disease), involve inflammation of the liver related to fat accumulation, and mimic alcoholic hepatitis but are observed in patients who seldom or never consume alcohol. NASH and NAFLD are frequently reported in both men and women, although it most often appears in women and is especially prevalent in the obese. Although the disease has been observed to be accompanied by several other pathological conditions, including diabetes mellitus, hyperlipidemia, hyperglycemia, all part of the “metabolic syndrome,” the cause and progression of the disease, as well as the causal or temporal relation to these conditions, is not well understood. However, in patients suffering from NAFLD and NASH in particular, certain characteristics of liver tissue and abnormalities of function are typical. Specifically, fatty deposits, tissue degeneration, inflammation, cell degeneration, cirrhosis, elevation of free fatty acids and other such abnormalities have come to be associated with nonalcoholic steatohepatitis and are frequently seen in patients suffering from forms of NAFLD.

Approximately 8% of patients who undergo liver biopsies will show histological evidence of NASH. The physiological condition that most commonly accompanies NASH is obesity, with approximately 70% and above of NASH sufferers also displaying clinically diagnosed obesity. NASH is particularly prevalent in obese patients who have undergone jejunal bypass to treat the obesity. In NASH patients, the extent of obesity tends to be generally correlated with the amount of steatosis and to be unrelated to non-insulin-dependent diabetes mellitis. However, non-insulin-dependent diabetes mellitis increases the prevalence of steatohepatitis especially in patients requiring insulin. Unless a massive (50-60%) amount of the excess body weight is eliminated, weight loss in patients before death does not appear to alleviate the steatosis and, somewhat paradoxically, obese patients who lost weight before death can have a higher incidence of steatohepatitis.

Even in NASH patients who do not consume any alcohol at all, liver biopsy specimens tend to mimic those seen in patients suffering from alcoholic hepatitis. However, a comparison of the two conditions reveals a higher incidence of vacuolation (indicative of diabetes) and steatosis in NASH as compared to alcoholic hepatitis. Patients suffering from alcoholic hepatitis also have a higher incidence of periportal and pericellular fibrosis and proliferation of the bile ducts. Overall, the symptoms and histological damage observed in alcoholic hepatitis patients are more severe than in NASH.

Currently, an established therapy for patients suffering from NASH does not exist. Weight loss is a common prescription, simply because obesity is frequently found in patients suffering from NASH. The effect of a reduction in weight loss on NASH cannot be determined with certainty, however, because obese patients seldom maintain significant weight reduction. Bariatric surgery with accompanying massive weight loss can decrease the histological appearance of NASH and NAFLD, but the long-term consequences of this approach to treatment are unknown and require delineation.

The present invention provides methods and compositions useful for the treatment and/or alleviation of NAFLD and NASH in particular, and the pharmaceutical formulations for their administration to a human. This application incorporates by reference U.S. Pat. No. 6,849,257 and U.S. Patent Application US20040223960.

### SUMMARY OF THE INVENTION

The present invention comprises methods and compositions for the treatment or alleviation of NAFLD (non-alcoholic fatty liver disease) and those conditions associated with NAFLD, including fatty liver disease, nonalcoholic steatohepatitis (NASH) and cirrhosis. Specifically, this invention is directed to pharmaceutical formulations of lysosomal acid lipase or related proteins and/or polypeptides as well as methods for their administration to a human suffering from NAFLD, as part of a treatment regimen to alleviate, or at least manage, the disease. This invention is also directed to a combination therapy treatment for treating The Metabolic Syndrome. The Metabolic Syndrome is a set of metabolic abnormalities including centrally distributed obesity, hyperlipidemia, elevated triglycerides, elevated blood pressure, Type II Diabetes and NAFLD. As part of a combination therapy regime for the treatment of The Metabolic Syndrome, pharmaceutical formulations of lysosomal acid lipase or related proteins and/or polypeptides are used as part of the combination therapy regime for treating the NAFLD (and NASH), component of The Metabolic Syndrome.

Generally, compositions used for practicing this invention include lipid (i.e., cholesteryl ester and mono-, di-, and tri-acylglycerols) hydrolyzing proteins or polypeptides, and in particular, the protein, lysosomal acid lipase (LAL).

Other lipid hydrolyzing proteins or polypeptides may also be used, such as proteins which show some sequence homology to lysosomal acid lipase or proteins having a Ser in similar active site locations to the Ser¹⁵³ residue in lysosomal acid lipase. Other proteins include polymorphic variants of lysosomal acid lipase with substitution of amino acid Pro(-6) to Thr and Gly2 to Arg and also polypeptides showing similar biological activity as lysosomal acid lipase, for example neutral and/or hormone sensitive cholesteryl esterase. Preferably, the composition comprises lysosomal acid lipase.

Exogenously produced lipid hydrolyzing proteins or polypeptides, contained in a pharmaceutically acceptable carrier, may be administered either orally, parenterally, by injection, intravenous infusion, inhalation, controlled dosage release or by intraperitoneal administration in order to diminish and/or eliminate atherosclerotic plaques. The preferred method of administration is by intravenous infusion.

Endogenously produced lipid hydrolyzing proteins and/or polypeptides may also be used for treatment of NAFLD. Generally, such a method involves providing a biologically active human lipid hydrolyzing protein or polypeptide, such as human lysosomal acid lipase, to cells of an individual suffering from NASH. This is accomplished by in vivo administration into cells competent for the production of biologically active human lipid hydrolyzing protein or polypeptide, a vector comprising and expressing a DNA sequence encoding biologically active human lipid hydrolyzing protein or polypeptide. The vector used may be a viral vector, including but not limited to a lentivirus, adenovirus, adeno-associated virus and virus-like vectors, a plasmid, or a lipid vesicle. The vector is taken up by the cells competent for the production of biologically active human lipid hydrolyzing protein or polypeptide. The DNA sequence is expressed and the biologically active human lipid hydrolyzing protein or polypeptide is produced. Additionally, the cells harboring this vector will secrete this biologically active lipid hydrolyzing protein or polypeptide which is then subsequently taken up by other cells.

Enhancement of naturally occurring enzymes, like LAL, may also be used to effect therapy. This could be accomplished by either inducing the production of LAL in situ from an existing resident gene, or, by stabilizing and improving intracellular localization, or, by enhancing the intrinsic catalytic activity with pharmacological chaperones. Such chaperones could be provided by a variety of exogenous routes including, but not limited to, intravenous, inhaled, subcutaneous, or oral, and be delivered to body tissues, e.g., the liver, to increase the resident/native lipid hydrolyzing function.

### BRIEF DESCRIPTION OF THE DRAWINGS

FIG. 1 depicts micrographs of liver sections from mice with low density lipoprotein receptor deficiency (ldlr−/−) who were fed a diet high in fat and cholesterol.

FIG. 2 depicts micrographs of liver sections from mice with ldlr−/− deficiency who had been on a high fat/high cholesterol diet and were administered a single iv dose of recombinant adenovirus containing the hLAL cDNA.

### DETAILED DESCRIPTION OF THE INVENTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

“About the same time” means that within about thirty minutes of administering one compound (nebivolol) to the patient, the other active compound(s) is/are administered to the patient. “About the same time” also includes simultaneous administration of the compounds.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the term “biologically active” refers to a protein having structural, regulatory, or biochemical functions of a naturally occurring molecule.

The phrase “cardiovascular agent” or “cardiovascular drug” refers to a therapeutic compound that is useful for treating or preventing a cardiovascular disease. Non-limiting examples of suitable cardiovascular agents include ACE inhibitors (angiotensin II converting enzyme inhibitors), ARB's (angiotensin II receptor antagonists), adrenergic blockers, adrenergic agonists, agents for pheochromocytoma, antianginal agents, antiarrhythmics, antiplatelet agents, anticoagulants, antihypertensives, antiinflammatory agents, calcium channel blockers, CETP inhibitors, COX-2 inhibitors, direct thrombin inhibitors, diuretics, endothelin receptor antagonists, HMG Co-A reductase inhibitors, inotropic agents, renin inhibitors, vasodilators, vasopressors, AGE crosslink breakers (advanced glycosylation end-product crosslink breakers, such as alagebrium, see U.S. Pat. No. 6,458,819), and AGE formation inhibitors (advanced glycosylation end-product formation inhibitors, such as pimagedine), and combinations thereof.

The term “combination therapy” refers to two or more different active agents which are administered at roughly about the same time (for example, where the active agents are in a single pharmaceutical preparation) or at different times (for example, one agent is administered to the subject before the other). The time difference may range from hours to days, but still constitutes a combination therapy technique.

The term “derivative,” as used herein, refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological function of the natural molecule. A derivative polypeptide is one modified, for instance by glycosylation, or any other process which retains at least one biological function of the polypeptide from which it was derived.

The terms “drug,” “pharmaceutically active agent,” “bioactive agent,” “therapeutic agent,” and “active agent” may be used interchangeably and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

The phrase “effective amount” refers to that amount of a substance that produces some desired local or systemic effect at a reasonable benefit/risk ratio applicable to any treatment. The effective amount of such substance will vary depending upon the subject and disease condition being treated, the weight and age of the subject, the severity of the disease condition, the manner of administration and the like, which can readily be determined by one of ordinary skill in the art.

As used herein, the term “exogenous lipid hydrolyzing proteins or polypeptides” refers to those produced or manufactured outside of the body and administered to the body; the term “endogenous lipid hydrolyzing proteins or polypeptides” refers to those produced or manufactured inside the body by some type of device (biologic, pharmacological chaperone, or other) for delivery to within or to other organs in the body.

The words “insertion” or “addition,” as used herein, refer to changes in an amino acid or nucleotide sequence resulting in the addition of one or more amino acid residues or nucleotides, respectively, to the sequence found in the naturally occurring molecule.

The term “Metabolic Syndrome” is used to define a set of conditions that places people at high risk for coronary artery disease. These conditions include Type II diabetes, central obesity also known as visceral adiposity, high blood pressure, and a poor lipid profile with elevated LDL (“bad”) cholesterol, low HDL (“good”) cholesterol, elevated triglycerides. All of these conditions are associated with high blood insulin levels. Associated diseases include NAFLD (especially in concurrent obesity) polycystic ovarian syndrome; hemochromatosis (iron overload); acanthosis nigricans (a skin condition featuring dark patches); Non-alcoholic steatohepatitis (NASH-an extreme form of fatty liver).

The term “NAFLD” (non-alcohol fatty liver disease) refers to a group of conditions where there is an accumulation of excess fat in the liver of people who drink little or no alcohol. The most common form of NAFLD is a condition called fatty liver disease. In fatty liver disease, fat accumulates in the liver cells. A small group of people with NAFLD may have a more serious condition termed non-alcoholic steatohepatitis (NASH). In NASH, fat accumulation is associated with liver cell inflammation and different degrees of scarring. Cirrhosis occurs when the liver sustains substantial damage, and the liver cells are gradually replaced by scar tissue which results in the inability of the liver to work properly. The use of the term “NAFLD” is used to include all conditions reflecting a form of non-alcohol fatty liver disease, but in particular, NASH.

The phrases “nucleic acid” or “nucleic acid sequence,” as used herein, refer to a nucleotide, oligonucleotide, polynucleotide, or any fragment thereof. These phrases also refer to DNA or RNA of genomic or synthetic origin which may be single-stranded or double-stranded and may represent the sense or the antisense strand, or to any DNA-like or RNA-like material. In this context, “fragments” refers to those nucleic acid sequences which, when translated, would produce polypeptides retaining some functional characteristic, e.g., lipase activity, or structural domain characteristic, of the full-length polypeptide.

The phrases “percent identity” or “percent homology” refers to the percentage of sequence similarity found in homologues of a particular amino acid or nucleic acid sequence when comparing two or more of the amino acid or nucleic acid sequences.

The term “pharmaceutically acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be acceptable in the sense of being compatible with the subject composition and its components and not injurious to the patient. Some examples of materials which may serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) IV fluids, including but not limited to Ringer's solution, 5% dextrose in water, and half normal saline; (19) ethyl alcohol; (20) phosphate buffer solutions; and (21) other non-toxic compatible substances employed in pharmaceutical formulations.

The term “Pharmacological Chaperones” refers to small molecules which stabilize the correct folding of a protein and are administered to the patient resulting in a recovery of function lost due to mutation.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “synthetic” is art-recognized and refers to production by in vitro chemical or enzymatic synthesis.

The phrase “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art.

The term “treating” is art-recognized and refers to curing as well as ameliorating at least one symptom of any condition or disease.

### Discussion

NAFLD and NASH in particular, involve the development of histologic changes in the liver that are comparable to those induced by excessive alcohol intake but in the absence of alcohol abuse. Macrovesicular and/or microvesicular steatosis, lobular and portal inflammation, and occasionally Mallory bodies with fibrosis and cirrhosis characterize NAFLD. NAFLD (and NASH in particular) is also commonly associated with hyperlipidemia, obesity, hypertension, and Type II diabetes mellitus, commonly known as The Metabolic Syndrome. Other clinical conditions characterized by hepatic steatosis and inflammation include excessive fasting, jejunoileal bypass, total parental nutrition, chronic hepatitis C, Wilson's disease, and adverse drug effects such as those from corticosteroids, calcium channel blockers, high dose synthetic estrogens, methotrexate and amiodarone. Thus, the term “nonalcoholic steatohepatitis” can be used to describe those patients who exhibit these biopsy findings, coupled with the absence of (a) significant alcohol consumption, (b) previous surgery for weight loss, (c) history of drug use associated with steatohepatitis, (d) evidence of genetic liver disease or (e) chronic hepatitis C infection. See, J. R. Ludwig et al., Mayo Clin. Proc., 55, 434 (1980) and E. E. Powell et al., Hepatol., 11, 74 (1990).

Hepatic steatosis (fatty liver) and steatohepatitis (fatty liver with inflammation or scarring) are two forms of the fatty liver disease that develops in children and adults, frequently associated with being overweight or obese. This disease develops in at least 10-20% of adolescents and adults that are obese and in 5-10% of those that are overweight. This type of liver disease is often referred to as non-alcoholic steatohepatitis (NASH), because it occurs in the absence of a significant amount of alcohol intake.

Suitable lipid hydrolyzing substances for use in this invention include, but are not limited to, glycoproteins such as LAL, homologues of LAL, wherein the homologues possess at least 85% sequence homology, due to degeneracy of the genetic code which encodes for LAL, polypeptides possessing similar biological activity to LAL and non-peptide derived substances. Also included are lipid hydrolyzing proteins and polypeptides which contain the catalytic lipase triad Asp-Ser-His, for example, where the Ser is a Ser¹⁵³ residue. Additional substances include polymorphic variants of LAL in which two of the amino acids are replaced with different amino acids. An example of such polymorphic variants are prepared by cloning LAL from normal human liver cDNA library and changing two nucleotides (C86 to A and G107 to A) which results in substitution of amino acid Pro(-6) to Thr and Gly2 to Arg in LAL, yielding four different polymorphic variants of LAL. Additional amino acid sequences include those capable of lipid hydrolysis, either catalytic or stoichiometric, wherein the residue 153 of the amino acid chain is a serine residue.

Further LAL-derived proteins include those proteins having the native LAL sequence, but which have more than six N-linked acetylglycosylation residues or fewer than six N-linked acetylglycosylation residues. Each glycosylation site has two N-linked acetylglucosamine residues, which are oligosaccharide-terminated, where the oligosaccharide-terminating residue can be, but is not limited to, an α-mannose, β-galactose, N-acetyl-neuraminic acid, N-acetyl-glucosamine or other receptor-recognized saccharide residues and where there are at least three oligosaccharide-terminating residues at each glycosylation site. The oligosaccharide may be recognized by a variety of receptors including but not limited to those for α-mannose, β-galactose, N-acetyl-neuraminic acid, N-acetyl-glucosamine and mannose-6-phosphate.

### Methods of Treatment of NAFLD Using Lipid Hydrolyzing Proteins

### Endogenous Therapy:

The principles of gene therapy for the production of therapeutic products within the body include the use of delivery vehicles (termed vectors) that can be non-pathogenic viral variants, lipid vesicles (liposomes), carbohydrate and/or other chemical conjugates of nucleotide sequences encoding the therapeutic protein or substance. These vectors are introduced into the body's cells by physical (direct injection), chemical or cellular receptor mediated uptake. Once within the cells, the nucleotide sequences can be made to produce the therapeutic substance within the cellular (episomal) or nuclear (nucleus) environments. Episomes usually produce the desired product for limited periods whereas nuclear incorporated nucleotide sequences can produce the therapeutic product for extended periods including permanently.

Such gene therapy approaches are used to produce therapeutic products for local (i.e., within the cell or organ) or distant beneficial effects. Both may provide decreases in pathologic effects and may combine to produce additive and/or synergistic therapy. For either effect, local or distant, the natural (termed normal) or altered (mutated) nucleotide sequences may be needed to enhance beneficial effects. The latter may be needed for targeted delivery to the specific cellular type involved in the pathology of the disease. For treatment of NASH, LAL would be delivered to the lysosomes of the liver cells.

An approach for the use of lipid removal substances, particularly lipid hydrolyzing proteins and polypeptides for the treatment of NAFLD, can be achieved by the gene therapy approaches discussed above. Such approaches provide a source of a biologically active human lipid hydrolyzing protein or polypeptide for delivery into the body by biologic or other production systems. This method of introduction can be achieved by internal or production sources (biologic or other, gene therapy vectors, liposomes, gene activation etc.) which lead to the production of biologically active human lipid hydrolyzing proteins and polypeptides by certain cells of the body. The source may provide for the local or distant supply by, for example, direct effects within the cell or by secretion out of the cells for delivery to other cells of the body, like those in atheromatous plaques. This includes, but is not limited to, somatic gene therapy approaches that would allow for the synthesis and/or otherwise production of the therapeutic substance in the body. In particular, nucleotide sequences encoding the functional, lipid hydrolyzing, sequences of the lysosomal acid lipase incorporated into conjugates, liposomes, viral (i.e., lentivirus, adenovirus, adeno-associated virus or other viruses or such virus-like vectors) vectors for expression of the active sequences for therapeutic effect. In addition, nucleotide sequences encompassing the functional components of biologic and therapeutic interest and residing in the body's cells could be made to produce, express or otherwise make the requisite compound in therapeutic amounts. The therapeutic lipid hydrolyzing protein or polypeptide, thus produced in the body, would lead to a reduction or elimination of the atheromatous plaques or other lesions of atherosclerotic plaques.

Variants and homologous nucleotide or encoded sequences of human lysosomal acid lipase incorporated for synthesis and/or production of the active protein/peptide are transiently or permanently integrated into cells for therapeutic production. The normal, polymorphic variants, specifically mutated or modified lysosomal acid lipase sequences may be expressed from the context of the vectors incorporated into cells for normal and/or specifically modified function to enhance or otherwise promote therapeutic effects.

Such sequences can lead to the in vivo synthesis of the desired biologically active human lysosomal acid lipase or other therapeutic proteins within cells after incorporation into cells by various routes as described above. Once within cells, the synthesized biologically active human lysosomal acid lipase or another therapeutic protein hydrolyzes cholesteryl esters and/or triglycerides within the lysosomes following their targeted delivery.

Additionally, human lysosomal acid lipase or other therapeutic human proteins or polypeptides produced from incorporated nucleotide sequences are secreted from cells, enter the circulatory system and are taken up by distant cells via receptor mediated endocytosis or other such delivery systems to the lysosomes or other appropriate subcellular compartments (e.g., endoplasmic reticulum, cytoplasm) of pathologically involved cells including but are not limited to, hepatocytes, other types liver cells, macrophages and smooth muscle cells. Lysosomal liberation of free cholesterol and/or fatty acids within such cells has at least two beneficial effects on hepatic fat accumulation by allowing: 1) free cholesterol and fatty acids to exit from the lysosome or other cellular compartment, and participate in the SREBP mediated down regulation of endogenous hepatocellular, macrophage or other cell type cholesterol and/or acylglycerol synthesis, and 2) free cholesterol and fatty acids to exit from the lysosome or other cellular compartment and be transported out of the cell by specific and non-specific transport mechanisms. Both effects are beneficial in reducing the amount of accumulated cholesteryl esters and acylglycerols within lysosomes and other cellular compartments of hepatocytes, other liver cell types, macrophages and/or other cells of within the liver.

The gene vectors containing the requisite nucleotide sequences or other components necessary for therapeutic expression are introduced into the body's cells by several routes as described above.

Endogenous therapy also contemplates the production of a protein or polypeptide where the cell has been transformed with a genetic sequence that turns on the naturally occurring gene encoding the protein, i.e., endogenous gene-activation techniques.

Pharmacological/chemical chaperones and/or inducers provide additional methods of increasing the available endogenous lipid hydrolyzing agent, i.e., LAL, in cells by enhancing the production of such from an existing natural gene or enhancing the function (by cellular location or intrinsic activity) of the natural hydrolyzing agent within the body (see U.S. Pat. Nos. 5,900,360; 6,270,954; 6,541,195; 6,599,919; 6,583,158; 6,589,964; which are all incorporated by reference). The principles of such approaches are known to those skilled in the art and include the use of chemical agents, administered by several exogenous routes (intravenous, oral, inhaled) that interact with the preexisting lipid hydrolyzing agent already existing within cells of the body or induced to product such. This interaction or induction leads to enhanced bioavailability/activity of the body's natural lipid hydrolyzing agent by increasing the amount, stability, intrinsic activity or cell localization for effective treatment.

### Exogenous Therapy:

The lipid hydrolyzing proteins or polypeptides useful in the present invention for exogenous therapy may be administered by any suitable means. One skilled in the art will appreciate that many suitable methods of administering the compound to a host in the context of the present invention, in particular a mammal, are available, and, although more than one route may be used to administer a particular protein or polypeptide, a particular route of administration may provide a more immediate and more effective reaction than another route.

Formulations suitable for administration by inhalation include aerosol formulations placed into pressurized acceptable propellants, such as dichlorodifluoromethane, propane, nitrogen, and the like. The active agent may be aerosolized with suitable excipients. For inhalation administration, the composition can be dissolved or dispersed in liquid form, such as in water or saline, preferably at a concentration at which the composition is fully solubilized and at which a suitable dose can be administered within an inhalable volume.

Formulations suitable for oral administration include (a) liquid solutions, such as an effective amount of the compound dissolved in diluents, such as water or saline, (b) capsules, sachets or tablets, each containing a predetermined amount of the active ingredient, as solids or granules, (c) suspensions in an appropriate liquid, and (d) suitable emulsions. Tablet forms may include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

Formulations suitable for intravenous infusion and intraperitoneal administration, for example, include aqueous and nonaqueous, isotonic sterile injection solutions, which can contain anti-oxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carriers for example, water, for injections, immediately prior to use. Extemporaneous injection solutions and suspensions can be prepared for sterile powders, granules, and tablets of the kind previously described.

Parenteral administration, if used, could also be by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained. See, e.g., U.S. Pat. No. 3,710,795, Higuchi, issued 1973, which is incorporated by reference herein.

The appropriate dosage administered in any given case will, of course, vary depending upon known factors, such as the pharmacodynamic characteristics of the particular protein or polypeptide and its mode and route of administration; the age, general health, metabolism, weight of the recipient and other factors which influence response to the compound; the nature and extent of the fatty liver disease; the kind of concurrent treatment; the frequency of treatment; and the effect desired.

The effective and/or appropriate dose of human LAL is administered on a regular basis by parenteral, transdermal, transmucosal or other exogenous routes, or by endogenous routes from vector expressing in a continuous or control manner LAL in hepatocytes, hematopoietic or other stem cells to supply the organ(s) of involvement. These latter include but are not limited to hepatocytes, macrophages, sinusoidal lining cells of the liver, and other cells of the body involved in the pathogenesis or expression of NASH or related disorders. The dosing would be determined and adjusted for body mass, and stage of disease to ensure adequate delivery of appropriate amount of supplemental LAL to tissues and cells to affect NASH or related disorders. Outcomes would be assessed by the return toward normalcy of the involved plasma and tissue biomarkers, including, but not limited to quantitative staging of tissue biopsies, glucose tolerance, and other appropriate markers. In particular, the decrease in hepatic fatty infiltration, fibrosis and inflammation would be assessed as treatment outcomes.

### Supporting Data:

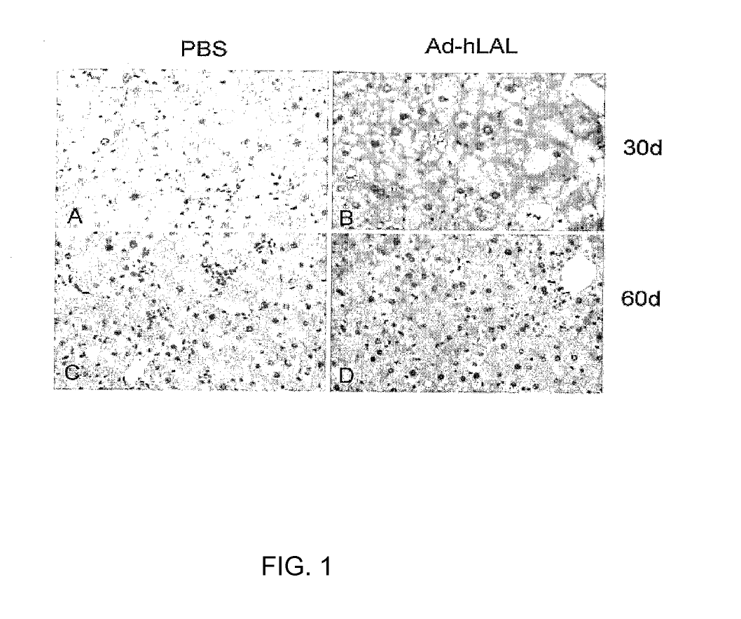

FIG. 1 shows micrographs of liver sections from mice with low density lipoprotein receptor deficiency (ldlr−/−) who were fed a diet high in fat and cholesterol, termed HFHC. In panel A, an untreated mouse on the HFHC diet for 2.5 months. Extensive micro- and macro-vacuolization of the hepatocytes and other cells is present. In addition, there is evidence of extensive inflammatory lesions. These findings are very similar to those found in human NASH/NAFLD. None of these findings are present in livers from ldlr−/− mice on regular chow diets. In panel B, ldlr−/− mice on the HFHC diet for 2.5 months received recombinantly produced human lysosomal acid lipase (hLAL) produced in Pichia pastoris yeast (phLAL) by parenteral injections for a period of 30 days. The micro- and macro-vesicular fat deposits are greatly diminished as is the inflammatory response.

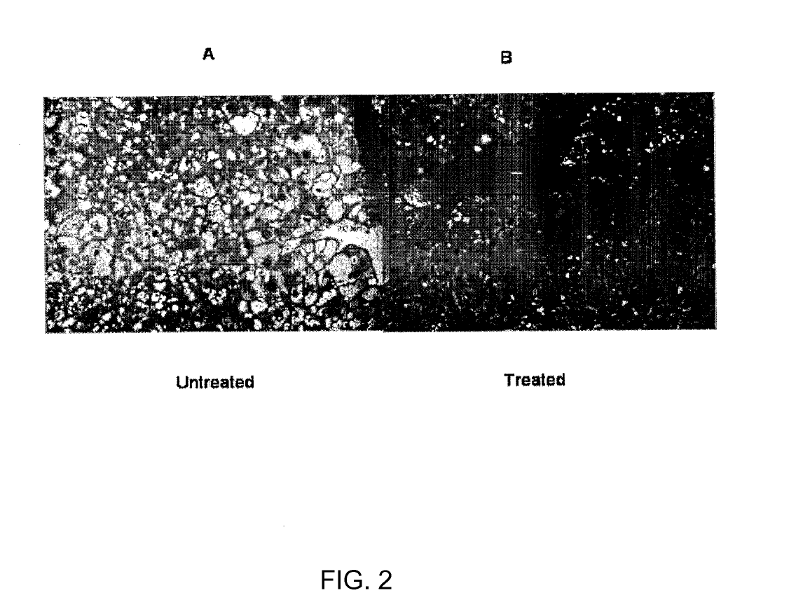

FIG. 2 show micrographs of liver sections from mice with ldlr−/− deficiency who had been on a high fat/high cholesterol diet. The panels under PBS were injected with phosphate buffered saline as a control and those under AdLAL were administered a single iv dose of recombinant adenovirus containing the hLAL cDNA. The upper panels in each are from males and the lower are from females. The PBS treated mice show fatty lesions similar to those in FIG. 1. In comparison, great reductions in fat deposition and inflammatory responses were present in the mice treated with the gene therapy vector, AdLAL.

Combination Therapy for the Use of Lipid Hydrolyzing Enzymes Such as LAL for Treatment of The Metabolic Syndrome

In one aspect, the present invention features a pharmaceutical composition comprising LAL or related and suitable lipid hydrolyzing protein, and at least one other active agent, used in the treatment of The Metabolic Syndrome. In a further embodiment, at least one of the active agents is a cardiovascular agent used in treating The Metabolic Syndrome. In a further embodiment, the at least one cardiovascular agent is selected from the group consisting of ACE inhibitors (angiotensin II converting enzyme inhibitors), ARB's (angiotensin II receptor antagonists), adrenergic blockers, adrenergic agonists, agents for pheochromocytoma, antiarrhythmics, antiplatelet agents, anticoagulants, antihypertensives, antiinflammatory agents, calcium channel blockers, CETP inhibitors, COX-2 inhibitors, direct thrombin inhibitors, diuretics, endothelin receptor antagonists, HMG Co-A reductase inhibitors, inotropic agents, rennin inhibitors, vasodilators, vasopressors, AGE crosslink breakers (advanced glycosylation end-product crosslink breakers, such as alagebrium, see U.S. Pat. No. 6,458,819), and AGE formation inhibitors (advanced glycosylation end-product formation inhibitors, such as pimagedine), and mixtures thereof. In one embodiment, the other cardiovascular agent is an ACE inhibitor or an ARB. In a further embodiment, the invention comprises at least two additional active agents, a cardiovascular agent and an antidiabetic.

In another aspect, the present invention features a method of treating a subject suffering from The Metabolic Syndrome, wherein the method comprises administering to the subject an effective amount of LAL or other related lipid-hydrolyzing enzyme, in combination with at least one other active agent, selected from the group consisting of cardiovascular agents, an antilipidemic agents, and/or an antidiabetic agents.

Non-limiting examples of the active agents that may be used in a combination therapy regime with LAL, or other related lipid hydrolyzing protein, (wherein the LAL is used to treat NAFLD) for the treatment of Metabolic Syndrome include, but are not limited to, the following representative classes of compounds, as well as their pharmaceutically acceptable salts, isomers, esters, ethers and other derivatives:

“Angiotensin I Converting Enzymes (ACE's) and Angiotensin II Receptor Antagonists (ARB's)”

“Angiotensin II receptor antagonists” (ARB's) are compounds which interfere with the activity of angiotensin II by binding to angiotensin II receptors and interfering with its activity. Angiotensin I and angiotensin II are synthesized by the enzymatic renin-angiotensin pathway. The synthetic process is initiated when the enzyme renin acts on angiotensinogen, a pseudoglobulin in blood plasma, to produce the decapeptide angiotensin I. Angiotensin I is converted by angiotensin converting enzyme (ACE) to angiotensin II (angiotensin-[1-8]octapeptide). The latter is an active pressor substance which has been implicated as a causative agent in several forms of hypertension in various mammalian species, e.g., humans.

Angiotensin II receptor antagonists (ARB's) are well known and include peptide compounds and non-peptide compounds. Most angiotensin II receptor antagonists are slightly modified congeners in which agonist activity is attenuated by replacement of phenylalanine in position 8 with some other amino acid; stability can be enhanced by other replacements that slow degeneration in vivo.

Examples of angiotensin II receptor antagonists include: peptidic compounds (e.g., saralasin and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4,5,6,7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl]1H-imidazole-5-yl[methylane]-2-thiophenepropanoic acid, Smith Kline Beecham Pharmaceuticals, Pa.); Losartan (DUP753/MK954, DuPont Merck Pharmaceutical Company); Remikirin (RO42-5892, F. Hoffman LaRoche A G); A2 agonists (Marion Merrill Dow) and certain non-peptide heterocycles (G. D. Searle and Company). Other non-limiting examples of ARBs include candesartan, eprosartan, irbesartan, losartan, and valsartan. Other ARBs may be identified using standard assaying techniques known to one of ordinary skill in the art.

“Angiotensin converting enzyme” (ACE) is an enzyme which catalyzes the conversion of angiotensin I to angiotensin II. ACE inhibitors include amino acids and derivatives thereof, peptides, including di- and tri-peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazepril (U.S. Pat. No. 4,410,520), phosphinylalkanoyl prolines such as fosinopril (U.S. Pat. No. 4,337,201) and trandolapril. Other non-limiting examples of ACE inhibitors include, but are not limited to, alacepril, benazepril, captopril, ceronapril, cilazapril, delapril, enalapril, enalaprilat, fosinopril, imidapril, lisinopril, perindopril, quinapril, ramipril, ramiprilat, spirapril, temocapril, trandolapril.

### Adrenergic Blockers

Non-limiting examples of adrenergic blockers, both α- and β-adrenergic blockers, that may be used in the compositions of the present invention include Beta-adrenergic receptor blockers include, but are not limited to, atenolol, acebutolol, alprenolol, befunolol, betaxolol, bunitrolol, carteolol, celiprolol, hydroxalol, indenolol, labetalol, levobunolol, mepindolol, methypranol, metindol, metoprolol, metrizoranolol, nebivolol, oxprenolol, pindolol, propranolol, practolol, sotalolnadolol, tiprenolol, tolamolol, timolol, bupranolol, penbutolol, trimepranol, yohimbine, 2-(3-(1,1-dimethylethyl)-amino-2-hydroxypropoxy)-3-pyridenecarbonitril HCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazole, 7-(2-hydroxy-3-t-butylaminpropoxy)phthalide. The above-identified compounds can be used as isomeric mixtures, or in their respective levorotating or dextrorotating form.

### Adrenergic Agonists

Non-limiting examples of adrenergic agonists, both α- and β-adrenergic agonists, that may be used in the compositions of the present invention include adrafinil, adrenalone, albuterol, amidephrine, apraclonidine, bitolterol, budralazine, carbuterol, clenbuterol, clonidine, clorprenaline, clonidine, cyclopentamine, denopamine, detomidine, dimetofrine, dioxethedrine, dipivefrin, dopexamine, ephedrine, epinephrine, etafedrine, ethylnorepinephrine, fenoterol, fenoxazoline, formoterol, guanabenz, guanfacine, hexoprenaline, hydroxyamphetamine, ibopamine, indanazoline, isoetharine, isometheptene, isoproterenol, mabuterol, mephentermine, metaproterenol, metaraminol, metizoline, methoxamine, methylhexaneamine, methoxyphenamine, midodrine, modafinil, moxonidine, naphazoline, norepinephrine norfenefrine, octodrine, octopamine, oxyfedrine, oxymetazoline, phenylephrine hydrochloride, phenylpropanolamine hydrochloride, phenylpropylmethylamine, pholedrine, pirbuterol prenalterol, procaterol, propylhexedrine, protokylol, pseudoephedrine, reproterol, rilmenidine, rimiterol, ritodrine, salmeterol, solterenol, synephrine, talipexole, terbutaline, tetrahydrozoline, tiamenidine, tramazoline, tretoquinol, tuaminoheptane, tulobuterol, tymazoline, tyramine, xamoterol, xylometazoline, and mixtures thereof.

### Antianginal Agents

Include but are not limited to amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, nitrates (including but not limited to glyceryl trinitrate (GTN, nitroglycerin, Nitro-Bid), isosorbide-5-mononitrate (5-ISMN, Ismo), amyl nitrate and nicorandil (Icorel)), primidolol, ranolazine hydrochloride, tosifen, verapamil hydrochloride).

### Antiarrhythmics

Non-limiting examples of antiarrhythmics that may be used in the compositions of the present invention include acebutolol, acecamide, adenosine, ajmaline, alprenolol, amiodarone, amoproxan, aprindine, aprotinolol, atenolol, azimilide, bevantolol, bidisomide, bretylium tosylate, bucumolol, butetolol, bunaftine, bunitrolol, bupranolol, butidrine hydrochloride, butobendine, capobenic acid, carazolol, carteolol, cifenline, cloranolol, disopyramide, dofetilide, encamide, esmolol, flecamide, hydroquinidine, ibutilide, indecamide, indenolol, ipratropium bromide, lidocaine, lorajmine, lorcamide, meobentine, mexiletine, moricizine, nadoxolol, nifenaolol, oxprenolol, penbutolol, pentisomide, pilsicamide, pindolol, pirmenol, practolol, prajmaline, procainamide hydrochloride, pronethalol, propafenone, propranolol, pyrinoline, quinidine, sematilide, sotalol, talinolol, tilisolol, timolol, tocamide, verapamil, viquidil, xibenolol, and mixtures thereof.

### Antiplatelet Agents

Non-limiting examples of antiplatelet agents that may be used in the compositions of the present invention include clopidogrel, dipyridamole, abciximab, and ticlopidine.

### Anticoagulants

Anti-coagulant agents are agents which inhibit the coagulation pathway by impacting negatively upon the production, deposition, cleavage and/or activation of factors essential in the formation of a blood clot. Non-limiting examples of anticoagulants (i.e. coagulation-related therapy) that may be used in the compositions of the present invention include Aggrenox, Agrylin, Amicar, Anturane, Arixtra, Coumadin, Fragmin, Heparin Sodium, Lovenox, Mephyton, Miradon, Persantine, Plavix, Pletal, Ticlid, Trental, Warfarin. Other “anti-coagulant” and/or “fibrinolytic” agents include Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant); rPro-UK; Abbokinase; Eminase; Streptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; reteplase; Trifenagrel; Warfarin; Dextrans.

Still other anti-coagulant agents include, but are not limited to, Ancrod; Anticoagulant Citrate Dextrose Solution; Anticoagulant Citrate Phosphate Dextrose Adenine Solution; Anticoagulant Citrate Phosphate Dextrose Solution; Anticoagulant Heparin Solution; Anticoagulant Sodium Citrate Solution; Ardeparin Sodium; Bivalirudin; Bromindione; Dalteparin Sodium; Desirudin; Dicumarol; Heparin Calcium; Heparin Sodium; Lyapolate Sodium; Nafamostat Mesylate; Phenprocoumon; Tinzaparin Sodium.

Inhibitors of platelet function are agents that impair the ability of mature platelets to perform their normal physiological roles (i.e., their normal function). Platelets are normally involved in a number of physiological processes such as adhesion, for example, to cellular and non-cellular entities, aggregation, for example, for the purpose of forming a blood clot, and release of factors such as growth factors (e.g., platelet-derived growth factor (PDGF)) and platelet granular components. One subcategory of platelet function inhibitors are inhibitors of platelet aggregation which are compounds which reduce or halt the ability of platelets to associate physically with themselves or with other cellular and non-cellular components, thereby precluding the ability of a platelet to form a thrombus.

Examples of useful inhibitors of platelet function include but are not limited to acadesine, anagrelide (if given at doses exceeding 10 mg/day), anipamil, argatroban, aspirin, clopidogrel, cyclooxygenase inhibitors such as nonsteroidal anti-inflammatory drugs and the synthetic compound FR-122047, danaparoid sodium, dazoxiben hydrochloride, diadenosine 5′,5′″-P1,P4-tetraphosphate (Ap4A) analogs, difibrotide, dilazep dihydrochloride, 1,2- and 1,3-glyceryl dinitrate, dipyridamole, dopamine and 3-methoxytyramine, efegatran sulfate, enoxaparin sodium, glucagon, glycoprotein IIb/IIIa antagonists such as Ro-43-8857 and L-700,462, ifetroban, ifetroban sodium, iloprost, Integrilin (eptifibatide), isocarbacyclin methyl ester, isosorbide-5-mononitrate, itazigrel, ketanserin and BM-13.177, lamifiban, lifarizine, molsidomine, nifedipine, oxagrelate, PGE, platelet activating factor antagonists such as lexipafant, prostacyclin (PGI₂), pyrazines, pyridinol carbamate, ReoPro (i.e., abciximab), sulfinpyrazone, synthetic compounds BN-50727, BN-52021, CV-4151, E-5510, FK-409, GU-7, KB-2796, KBT-3022, KC-404, KF-4939, OP-41483, TRK-100, TA-3090, TFC-612 and ZK-36374,2,4,5,7-tetrathiaoctane, 2,4,5,7-tetrathiaoctane 2,2-dioxide, 2,4,5-trithiahexane, theophylline, pentoxifylline, thromboxane and thromboxane synthetase inhibitors such as picotamide and sulotroban, ticlopidine, tirofiban, trapidil and ticlopidine, trifenagrel, trilinolein, 3-substituted 5,6-bis(4-methoxyphenyl)-1,2,4-triazines, and antibodies to glycoprotein IIb/IIIa as well as those disclosed in U.S. Pat. No. 5,440,020, and anti-serotonin drugs, Clopidogrel; Sulfinpyrazone; Aspirin; Dipyridamole; Clofibrate; Pyridinol Carbamate; PGE; Glucagon; Antiserotonin drugs; Caffeine; Theophylline Pentoxifylline; Ticlopidine.

### Antihypertensives

Non-limiting examples of antihypertensives that may be used in the compositions of the present invention include amlodipine, benidipine, benazepril, candesartan, captopril, darodipine, diltiazem HCl, diazoxide, doxazosin HCl, enalapril, eprosartan, losartan mesylate, felodipine, fenoldopam, fosinopril, guanabenz acetate, irbesartan, isradipine, lisinopril, mecamylamine, minoxidil, nicardipine HCl, nifedipine, nimodipine, nisoldipine, phenoxybenzamine HCl, prazosin HCl, quinapril, reserpine, terazosin HCl, telmisartan, and valsartan.

This invention also contemplates fixed dose combinations of nebivolol with hydrochlorothiazide and at least one other additional active agent.

### Antilipemic Agents

Non-limiting examples of antilipemic agents that may be used in the compositions of the present invention include acipimox, aluminum nicotinate, atorvastatin, cholestyramine resin, colestipol, polidexide, beclobrate, fluvastatin, gemfibrozil, lovastatin, icofibrate, niacin; PPARα agonist such as fibrates, which include, but are not limited to fenofibrate, clofibrate, pirifibrate, ciprofibrate, bezafibrate, clinofibrate, ronifibrate, theofibrate, clofibric acid, etofibrate, and gemfibrozil; pravastatin sodium, simfibrate, simvastatin, niceritrol, nicoclonate, thyropropic acid, thyroxine, acifran, azacosterol, benfluorex, beta-benzalbutyramide, carnitine, chondroitin sulfate clomestrone, detaxtran, dextran sulfate sodium, 5, 8, 11, 14, 17-eicosapentaenoic acid, eritadenine, furazabol, meglutol, melinamide, mytatrienediol, ornithine, gamma-oryzanol, pantethine, pentaerythritol tetraacetate, alpha-phenylbutyramide, pirozadil, probucol, beta-sitosterol, sultosilic acid (piperazine salt), tiadenol, triparanol, xenbucin, and mixtures thereof.

### Antidiabetics

Non-limiting examples of antidiabetics that may be used in the compositions of the present invention include biguanides such as buformin, metformin, and phenformin; hormones such as insulin; sulfonylurea derivatives such as acetohexamide, 1-butyl-3-metanilylurea, carbutamide, chlorpropamide, glibornuride, gliclazide, glimepiride, glipizide, gliquidone, glisoxepid, glyburide, glybuthiazole, glybuzole, glyhexamide, glymidine, glypinamide, phenbutamide, tolazamide, tolbutamide, tolcyclamide; HDL agonists; PPARγ agonists such as thiazolidinediones such as pioglitazone, rosiglitazone, and troglitazone; and others including acarbose, calcium mesoxalate, miglitol, and repaglinide.

### Calcium Channel Blockers

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogeneous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Non-limiting examples of calcium channel blockers that may be used in the compositions of the present invention include bepridil, clentiazem, diltiazem, fendiline, gallopamil, mibefradil, prenylamine, semotiadil, terodiline, verapamil, amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, perhexyline, and mixtures thereof.

### COX-2 Inhibitors

Non-limiting examples of COX-2 inhibitors that may be used in the compositions of the present invention include compounds according to the following: all of the compounds and substances beginning on page 8 of Winokur WO99/20110 as members of three distinct structural classes of selective COX-2 inhibitor compounds, and the compounds and substances which are selective COX-2 inhibitors in Nichtberger, U.S. Pat. No. 6,136,804, Oct. 24, 2000, entitled “Combination therapy for treating, preventing, or reducing the risks associated with acute coronary ischemic syndrome and related conditions”, and the compounds and substances which are selective COX-2 inhibitors in Isakson et al, PCT application WO/09641645 published Dec. 27, 1996, filed as PCT/US 9509905 on Jun. 12, 1995, entitled “Combination of a Cyclooxygenase-2 Inhibitor and a Leukotriene B4 Receptor Antagonist for the Treatment of Inflammations.” The meaning of COX-2 inhibitor in this invention shall include the compounds and substances referenced and incorporated into Winokur WO99/20110 by reference to art therein, the compounds and substances referenced and incorporated into Nichtberger, U.S. Pat. No. 6,136,804, Oct. 24, 2000, by reference to art therein, and the compounds and substances which are COX-2 inhibitors referenced and incorporated into Isakson et al, PCT application WO/09641645 published Dec. 27, 1996, filed as PCT/US 9509905 on Jun. 12, 1995, entitled “Combination of a Cyclooxygenase-2 Inhibitor and a Leukotriene B4 Receptor Antagonist for the Treatment of Inflammations.” The meaning of COX-2 inhibitor in this invention also includes rofecoxib, and celecoxib, marketed as VIOXX and CELEBREX by Merck and Searle/Pfizer respectively. Rofecoxib is discussed in Winokur, WO99/20110 as compound 3, on p. 9. Celecoxib is discussed as SC-58635 in the same reference, and in T. Penning, Synthesis and biological evaluation of the 1,5-diarylpyrazole class of cyclooxygenase-2 inhibitors: identification of 4-[5-(4-methylphenyl)-3-(trifluoromethyl)-1H-pyrozol-1-yl]benzenesulfonami de (SC-58635, celecoxib)”, J. Med. Chem. Apr. 25, 1997: 40(9): 1347-56. The meaning of COX-2 inhibitor in this invention also includes SC299 referred to as a fluorescent diaryloxazole. C. Lanzo et al, “Fluorescence quenching analysis of the association and dissociation of a diarylheterocycle to cyclooxygenasel-1 and cyclooxygenase-2: dynamic basis of cycloxygenase-2 selectivity”, Biochemistry May 23, 2000, vol. 39(20):6228-34, and in J. Talley et al, “4,5-Diaryloxazole inhibitors of cyclooxygenase-2 (COX-2)”, Med. Res. Rev. May 1999; 19(3): 199-208. The meaning of COX-2 inhibitor in this invention also includes valdecoxib, See, “4[5-Methyl-3-phenylisoxazol-1-yl]benzenesulfonamide, Valdecoxib: A Potent and Selective Inhibitor of COX-2”, J. Med. Chem. 2000, Vol. 43: 775-777, and parecoxib, sodium salt or parecoxib sodium, See, N-[[(5-methyl-3-phenylixoxazol-4-yl)-phenyl]sulfonyl]propanimide, Sodium Salt, Parecoxib Sodium: A Potent and Selective Inhibitor of COX-2 for Parenteral Administration”, J. Med. Chem. 2000, Vol. 43: 1661-1663. The meaning of COX-2 inhibitor in this invention also includes the substitution of the sulfonamide moiety as a suitable replacement for the methylsulfonyl moiety. See, J. Carter et al, Synthesis and activity of sulfonamide-substituted 4,5-diaryl thiazoles as selective cyclooxygenase-2 inhibitors.” Bioorg. Med. Chem. Lett Apr. 19, 1999: Vol. 9(8): 1171-74, and compounds referenced in the article “Design and synthesis of sulfonyl-substituted 4,5-diarylthiazoles as selective cyclooxygenase-2 inhibitors”, Bioorg. Med. Chem. Lett Apr. 19, 1999: Vol. 9(8): 1167-70. The meaning of this invention includes a COX-2 inhibitor, NS398 referenced in two articles: Attiga et al, “Inhibitors of Prostaglandin Synthesis Inhibit Human Prostate Tumor Cell Invasiveness and Reduce the Release of Matrix Metalloproteinases”, 60 Cancer Research 4629-4637, Aug. 15, 2000, and in “The cyclooxygenase-2 inhibitor celecoxib induces apoptosis by blocking Akt activation in human prostate cancer cells independently of Bcl-2,” Hsu et al, 275(15) J. Biol. Chem. 11397-11403 (2000). The meaning of COX-2 inhibitor in this invention includes the cyclo-oxygenase-2 selective compounds referenced in Mitchell et al, “Cyclo-oxygenase-2: pharmacology, physiology, biochemistry and relevance to NSAID therapy”, Brit. J. of Pharmacology (1999) vol. 128: 1121-1132, see especially p. 1126. The meaning of COX-2 inhibitor in this invention includes so-called NO-NSAIDs or nitric oxide-releasing-NSAIDs referred to in L. Jackson et al, “COX-2 Selective Nonsteroidal Anti-Inflammatory Drugs: Do They Really Offer Any Advantages?”, Drugs, June, 2000 vol. 59(6): 1207-1216 and the articles at footnotes 27, and 28. Also included in the meaning of COX-2 inhibitor in this invention includes any substance that selectively inhibits the COX-2 isoenzyme over the COX-1 isoenzyme in a ratio of greater than 10 to 1 and preferably in ratio of at least 40 to 1 as referenced in Winokur WO 99/20110, and has one substituent having both atoms with free electrons under traditional valence-shell-electron-pair-repulsion theory located on a cyclic ring (as in the sulfylamine portion of celecoxib), and a second substituent located on a different ring sufficiently far from said first substituent to have no significant electron interaction with the first substituent. The second substituent should have an electronegativity within such substituent greater than 0.5, or the second substituent should be an atom located on the periphery of the compound selected from the group of a halogen F, Cl, Br or I, or a group VI element, S or O. Thus for purposes of this last included meaning of a COX-2 inhibitor, one portion of the COX-2 inhibitor should be hydrophilic and the other portion lipophilic. Also included as a COX-2 inhibitor are compounds listed at page 553 in Pharmacotherapy: A Pathophysiologic Approach, Depiro et al (McGraw Hill 1999) including nabumetone and etodolac. Recognizing that there is overlap among the selective COX-2 inhibitors set out in this paragraph, the intent of the term COX-2 inhibitor is to comprehensively include all selective COX-2 inhibitors, selective in the sense of inhibiting COX-2 over COX-1. The inventors add to the class of COX-2 inhibitors useful in the invention the drug bearing the name etoricoxib referenced in the Wall Street Journal, Dec. 13, 2000, manufactured by Merck. See, also, Chauret et al., “In vitro metabolism considerations, including activity testing of metabolites, in the discovery and selection of the COX-2 inhibitor etoricoxib (MK-0663),” Bioorg. Med. Chem. Lett. 11(8): 1059-62 (Apr. 23, 2001). Another selective COX-2 inhibitor is DFU [5,5-dimethyl-3-(3-fluorophenyl)-4-(4-methylsulphonyl) phenyl-2(5H)-furanone] referenced in Yergey et al, Drug Metab. Dispos. 29(5):638-44 (May 2001). The inventors also include as a selective COX-2 inhibitor the flavonoid antioxidant silymarin, and an active ingredient in silymarin, silybinin, which demonstrated significant COX-2 inhibition relative to COX-1 inhibition. The silymarin also showed protection against depletion of glutathione peroxidase. Zhao et al, “Significant Inhibition by the Flavonoid Antioxidant Silymarin against 12-O-tetracecanoylphorbol 13-acetate-caused modulation of antioxidant and inflammatory enzymes, and cyclooxygenase 2 and interleukin-1 alpha expression in SENCAR mouse epidermis: implications in the prevention of stage I tumor promotion,” Mol. Carcinog. December 1999, Vol 26(4):321-33 PMID 10569809. Silymarin has been used to treat liver diseases in Europe.

A number of the above-identified COX-2 inhibitors are prodrugs of selective COX-2 inhibitors, and exert their action by conversion in vivo to the active and selective COX-2 inhibitors. The active and selective COX-2 inhibitors formed from the above-identified COX-2 inhibitor prodrugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present invention.

### “Direct Thrombin Inhibitors”

Non limiting examples of direct thrombin inhibitors include hirudin, hirugen, Hirulog, argatroban, PPACK, and thrombin aptamers.

### Diuretics

Non-limiting examples of diuretics that may be used in the compositions of the present invention include althiazide, bendroflumethiazide, benzthiazide, buthiazide, chlorthalidone, cyclopenthiazide, cyclothiazide, epithiazide, ethiazide, fenquizone, indapamide, hydroflumethiazide, methyclothiazide, meticrane, metolazone, paraflutizide, polythiazide, quinethazone, teclothiazide, trichloromethiazide, chlormerodrin, meralluride, mercamphamide, mercaptomerin sodium, mercumatilin sodium, mercurous chloride, mersalyl, acefylline, 7-morpholinomethyl-theophylline, pamabrom, protheobromine, theobromine, canrenone, oleandrin, spironolactone, acetazolamide, ambuside, azosemide, bumetanide, butazolamide, clopamide, clorexolone, disulfamide, ethoxzolamide, furosemide, mefruside, methazolamide, piretanide, torsemide, tripamide, xipamide, aminometradine, amisometradine, amanozine, amiloride, arbutin, chlorazanil, ethacrynic acid, etozolin, hydracarbazine, isosorbide, mannitol, metochalcone, muzolimine, perhexyline, ticrynafen, triamterene, urea, and mixtures thereof. Depending on the diuretic employed, potassium may also be administered to the patient in order to optimize the fluid balance while avoiding hypokalemic alkalosis. The administration of potassium can be in the form of potassium chloride or by the daily ingestion of foods with high potassium content such as, for example, bananas or orange juice.

### HMG-CoA Reductase Inhibitor (Statins)

HMG-CoA (3-hydroxy-3-methylglutaryl-coenzyme A) reductase is the microsomal enzyme that catalyzes the rate limiting reaction in cholesterol biosynthesis (HMG-CoA6Mevalonate). An HMG-CoA reductase inhibitor inhibits HMG-CoA reductase, and as a result inhibits the synthesis of cholesterol. A number of HMG-CoA reductase inhibitors have been used to treat individuals with hypercholesterolemia. More recently, HMG-CoA reductase inhibitors have been shown to be beneficial in the treatment of stroke (Endres M, et al., Proc Natl Acad Sci USA, 1998, 95:8880-5).

HMG-CoA reductase inhibitors useful for co-administration with the agents of the invention include, but are not limited to, simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. Nos. 5,622,985; 5,135,935; 5,356,896; 4,920,109; 5,286,895; 5,262,435; 5,260,332; 5,317,031; 5,283,256; 5,256,689; 5,182,298; 5,369,125; 5,302,604; 5,166,171; 5,202,327; 5,276,021; 5,196,440; 5,091,386; 5,091,378; 4,904,646; 5,385,932; 5,250,435; 5,132,312; 5,130,306; 5,116,870; 5,112,857; 5,102,911; 5,098,931; 5,081,136; 5,025,000; 5,021,453; 5,017,716; 5,001,144; 5,001,128; 4,997,837; 4,996,234; 4,994,494; 4,992,429; 4,970,231; 4,968,693; 4,963,538; 4,957,940; 4,950,675; 4,946,864; 4,946,860; 4,940,800; 4,940,727; 4,939,143; 4,929,620; 4,923,861; 4,906,657; 4,906,624 and 4,897,402, the disclosures of which patents are incorporated herein by reference.

Other non-limiting examples of HMG-CoA reductase inhibitors that may be used in the compositions of the present invention include mevastatin, pitavastatin, rosuvastatin, gemcabene, and probucol.

### Inotropic Agents

Non-limiting examples of inotropic agents that may be used in the compositions of the present invention include acefylline, acetyldigitoxins, 2-amino-4-picoline, amrinone, benfurodil hemisuccinate, bucladesine, camphotamide, convallatoxin, cymarin, denopamine, deslanoside, digitalin, digitalis, digitoxin, digoxin, dobutamine, docarpamine, dopamine, dopexamine, enoximone, erythrophleine, fenalsomine, gitalin, gitoxin, glycocyamine, heptaminol, hydrastinine, ibopamine, lanatosides, loprinine, milrinone, nerifolin, oleandrin, ouabain, oxyfedrine, pimobendan, prenalterol, proscillaridin, resibufogenin, scillaren, scillarenin, strophanthin, sulmazole, theobromine, vesnarinone, xamoterol, and mixtures thereof.

### Vasodilators

Non-limiting examples of vasodilators that may be used in the compositions of the present invention include bencyclane, cinnarizine, citicoline, cyclandelate, ciclonicate, diisopropylamine dichloroacetate, eburnamonine, fasudil, fenoxedil, flunarizine, ibudilast, ifenprodil, isosorbide dinitrate, isosorbide mononitrate, lomerixine, nafronyl, nicametate, nicergoline, nimodipine, papaverine, pentifylline, tinofedrine, vancamine, vinpocetine, viquidil, amotriphene, bendazol, benfurodil hemisuccinate, benziodarone, chloracizine, chromonar, clobenfurol, clonitrate, cloricromen, dilazep, dipyridamole, droprenilamine, efloxate, erythrityl tetranitrate, etafenone, fendiline, floredil, ganglefence, heart muscle extract, hexestrol bis(alpha-diethylaminoethyl ether), hexobendine, hydralazine compound, lidoflazine, mannitol hexanitrate, medibazine, nitroglycerin, isosorbide mononitrate, isosorbide dinitrate, and other nitrates, pentaerythritol tetranitrate, pentrinitrol, perhexyline, pimefylline, prenylamine, propatyl nitrate, pyridofylline, trapidil, tricromyl, trimetazidine, trolnitrate phosphate, visnadine, aluminum nicotinate, bamethan, bencyclane, betahistine, bradykinin, brovincamine, bufeniode, buflomedil, butalamine, cetiedil, ciclonicate, cinepazide, cinnarizine, cyclandelate, diisopropylamine dichloroacetate, eledoisin, fenoxedil, flunazine, hepronicate, ifenprodil, iloprost, inositol niacinate, isoxsuprine, kallidin, kallikrein, moxisylyte, nafronyl, nicametate nicergoline, nicofuranose, nicotinyl alcohol, nylidrin, pentifylline, pentoxifylline, piribedil, prostaglandin E1, suloctidil, tolazoline, xanthinol niacinate, and mixtures thereof.

Note that “hydralazine compound” refers to a compound having the formula:

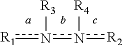

wherein a, b and c are each independently a single or a double bond; R₁ and R₂ are each independently a hydrogen, an alkyl, an ester or a heterocyclic ring; R₃ and R₄ are each independently a lone pair of electrons or a hydrogen, with the proviso that at least one of R₁, R₂, R₃ and R₄ is not a hydrogen. Examples of hydralazine compounds include, but are not limited to budralazine, cadralazine, dihydralazine, endralazine, hydralazine, pildralazine, todralazine and the like.

### Vasopressors

Non-limiting examples of vasopressors that may be used in the compositions of the present invention include amezinium methyl sulfate, angiotensin amide, dimetofrine, dopamine, etifelmin, etilefrin, gepefrine, metaraminol, methoxamine, midodrine, norepinephrine, pholedrine, synephrine, and mixtures thereof.

### AGE Crosslink Breakers (Advanced Glycosylation End-Product Crosslink Breakers)

Non-limiting examples of AGE crosslink breakers that may be used in the compositions of the present invention include Alagebrium.

AGE Formation Inhibitors (advanced glycosylation end-product formation inhibitors)

Non-limiting examples of AGE formation inhibitors that may be used in the compositions of the present invention include Pimagedine.

### Dosages

Administration of the compositions of the present invention will be in an amount sufficient to achieve a therapeutic effect as recognized by one of ordinary skill in the art.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the co-active compounds will generally be in the range of about 0.01 ng to about 1 g per kg body weight, specifically in the range of about 1 ng to about 0.1 g per kg, and more specifically in the range of about 100 ng to about 10 mg per kg body weight.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the steroidal anti inflammatory drug) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

In general, the doses of an active agent will be chosen by a physician based on the age, physical condition, weight and other factors known in the medical arts.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein.

1. A method for treatment of NAFLD in a mammal comprising administering to said mammal a safe and effective amount of lysosomal acid lipase, sufficient to treat said condition.

2. (canceled)

3. (canceled)

4. The method of claim 2 wherein said lysosomal acid lipase targets a receptor site for uptake into cells.

5. The method of claim 4 wherein said receptor site is selected from the group consisting of oligosaccharide recognition receptors and peptide sequence recognition receptors.

6. The method of claim 5 wherein said receptor site is a mannose receptor site.

7. The method of claim 2 wherein the lysosomal acid lipase is exogenously produced.

8. The method of claim 7 wherein said lysosomal acid lipase is in a pharmaceutically acceptable carrier and is administered either orally, parenterally, by injection, intravenous infusion, inhalation, controlled dosage release or by intraperitoneal administration.

9. The method of claim 8 wherein the lysosomal acid lipase is administered by intravenous infusion.

10. The method of claim 2 wherein the lysosomal acid lipase has fewer than six N-linked acetylglycosylation residues.

11. The method of claim 10 wherein the N-acetylglycosylation residue is oligosaccharide-terminated.

12. The method of claim 11 wherein the oligosaccharide terminating residue is a mannose residue.

13. The method of claim 2 wherein the lysosomal acid lipase has more than six N-linked acetylglycosylation residues.

14. The method of claim 13 wherein the N-acetylglycosylation residue is oligosaccharide-terminated.

15. The method of claim 14 wherein the oligosaccharide terminating residue is a mannose residue.

16-28. (canceled)

29. A method for treatment of NASH in a mammal comprising administering to said mammal a safe and effective amount of exogenously produced lysosomal acid lipase sufficient to treat said condition.

30. The method of claim 29 wherein the lysosomal acid lipase is in a suitable pharmaceutically acceptable carrier.

31. The method of claim 30 wherein the lysosomal acid lipase is administered by intravenous infusion.

32-41. (canceled)
